(19) United States
(12) Patent Application Publication
(10) Pub. No.: US 2019/0117710 A1
(43) Pub. Date: Apr. 25, 2019

(54) METHODS FOR REGULATING ENDOGENOUS PRODUCTION OF CHECKPOINT PROTEINS

(71) Applicant: Wyvern Pharmaceutical Inc., Calgary (CA)

(72) Inventors: Jill L. THOMPSON, Calgary (CA); Bradley G. THOMPSON, Calgary (CA)

(73) Assignee: Wyvern Pharmaceutical Inc.

(21) Appl. No.: 15/789,266

(22) Filed: Oct. 20, 2017

(51) Int. Cl. A61K 35/76; C12N 15/117

(52) U.S. Cl. CPC A61K 35/76; C12N 2310/17; C12N 15/117

(57) ABSTRACT

The present disclosure relates to one or more agents, therapies, treatments, and methods of use of the agents and/or therapies and/or treatments for upregulating production and/or functionality of one or more immune checkpoint molecules. Embodiments of the present disclosure can be used as a therapy or a treatment for a subject that has a condition whereby the subject's immune system is or is likely to become, dysregulated.

### TECHNICAL FIELD

The present disclosure generally relates to molecules that act as checkpoint molecules within a subject. In particular, the present disclosure relates to agents, therapies, and methods of use of the agents and/or therapies for upregulating production of one or more of the checkpoint molecules as a therapy or treatment of conditions that the subject has experienced or will experience dysregulation of their immune system.

### BACKGROUND

The immune system has evolved to allow an individual to differentiate between self and foreign matter. A number of cascades of signaling molecules and immune cells are well characterized for their ability to recognize foreign matter and for calling upon the production and stimulation of effector cells of the immune system to kill, break down, consume, or sheath the foreign matter in order to protect a host.

It is known that under various conditions the immune system can become dysregulated. A dysregulated immune system can cause further damage to the host thereby preventing healing, and may result in a loss of homeostatic controls and/or a chronically stimulated immune system.

Immune checkpoint molecules, in particular checkpoint proteins (CPP) are known participants in the immune system's responses to foreign matter. CPP can be categorized as stimulatory or inhibitory. Many stimulatory CPP have been identified as participating, either directly or indirectly, in increasing a host's immune response or protecting cells from the host's immune response. Many inhibitory CPP have also been identified that function to participate, either directly or indirectly, in decreasing a host's immune response. For example, it is known that exogenous addition of a single CPP to cell surfaces may have therapeutic benefit in conditions were the immune system is dysregulated.

Known approaches to conditions where the immune system is dysregulated are the commercially available pharmaceutical products Abatacept and Belatacept. These products can act as an exogenous mimic of inhibitory CPP. For example, Abatacept is composed of the fragment crystallizable (Fc) portion of an immunoglobulin G1 (IgG1) that is linked to an extracellular domain of cytotoxic T-lymphocyte-associated protein 4 (CTLA-4). Mechanistically, Abatacept and Belatacept interfere with antigen-presenting cells' ability to generate a co-stimulatory signal that is necessary to activate T-cells. Abatacept is used to treat rheumatoid arthritis and juvenile idiopathic arthritis. Abatacept has also been shown to be efficacious in psoriasis (phase 1 study results). Belatacept is used in renal transplant patients. The results indicate that Belatacept may be as efficacious as cyclosporine at decreasing the immune reaction. Belatacept has also been shown to be efficacious in rheumatoid arthritis (phase 2 study results).

The use of exogenous mimics of a single inhibitory CPP may be limited by the effectiveness of the exogenous treatment to access a subject's cells and activate the immune protection or downregulation functions of the subject's cells. Furthermore, the known approaches are directed at mimicking individual inhibitory CPP.

### SUMMARY

Some embodiments of the present disclosure relate to a method of making an agent/target cell complex, the method comprises a step of administering a therapeutically effective amount of the agent to a subject, wherein the agent/target cell complex increases the subject's production and/or functionality of one or more checkpoint protein(s) (CPP).

Some embodiments of the present disclosure relate to a method of making an agent/target cell complex, the method comprising a step of administering a sufficient amount of an agent to a target cell whereby the agent/target cell complex is formed, wherein the agent/target cell complex increases the production and/or functionality of one or more checkpoint protein(s) (CPP) by said target cell.

Some embodiments of the present disclosure relate to a pharmaceutical agent that comprises an agent, a pharmaceutically acceptable carrier, and/or an excipient. The agent may upregulate production or functionality of a CPP and/or a regulatory molecule that upregulates the production or functionality of a CPP.

Some embodiments of the present disclosure relate to a kit used for treatment of a condition or for delivery of a therapy to a subject. The kit comprises a unit dosage of an agent, a carrier for the unit dosage, and instructions for administering the unit dosage to the subject. The agent may upregulate production or functionality of a CPP and/or a regulatory molecule that upregulates the production of or functionality of a CPP. The carrier may be a solid carrier such as a pill or tablet or a liquid. The instructions may describe how the solid carrier may be administered to a subject for an optimal effect. The instructions may also describe how the liquid carrier may be administered to a subject by various routes of administration.

Some embodiments of the present disclosure relate to a method of treating a condition. The method comprises a step of administering to a subject a therapeutically effective amount of an agent that upregulates a production and/or a functionality of one or more CPP.

Some embodiments of the present disclosure relate to a use of an agent for treating a condition, wherein the agent upregulates a production and/or a functionality of one or more CPP in a subject that receives the agent.

Some embodiments of the present disclosure relate to a method for upregulating a production and/or a functionality of one or more CPP, the method comprising a step of making an agent/target cell complex.

An agent that preferentially induces endogenous production of one or more inhibitory CPP, for example by upregulating the presence of inhibitory CPP on the subject's cellular surfaces, may be more effective at masking the target cells from the immune system than an exogenously added single inhibitory CPP or single inhibitory CPP mimic.

Embodiments of the present disclosure relate to at least two approaches for inducing endogenous production of one or more inhibitory CPP. A first approach utilizes chronic viral infections, or an attenuated virus, that may upregulate CPP expression on infected target-cell surfaces and “hide” those target cells from the subject's immune system. Infecting patients with these viruses would result in increased endogenous inhibitory CPP production on target-cell surfaces. A second approach utilizes gene vectors containing nucleotide sequences and/or genes for one or more inhibitory CPP (CTLA-4, PD-1, PD-L1, PD-L2, IDO1) and/or regulatory molecules of an inhibitory CPP (such as gamma interferon also referred to herein as y interferon) can be administered to subjects to increase the production and/or functionality of endogenous CPP on cell surfaces.

Without being bound by any particular theory, embodiments of the present disclosure may be useful for treating conditions where the subject's immune system has become dysregulated, for example during an autoimmune disease, and for decreasing the subject's immune response to foreign matter such as surgically transplanted grafts or organs and surgically implanted medical devices and diagnostic devices.

### DETAILED DESCRIPTION

### Definitions

Unless defined otherwise, all technical and scientific terms used herein have the meanings that would be commonly understood by one of skill in the art in the context of the present specification. Although any methods and materials similar or equivalent to those described herein can also be used in the practice or testing of the present disclosure, the preferred methods and materials are now described. All publications mentioned herein are incorporated herein by reference to disclose and describe the methods and/or materials in connection with which the publications are cited.

As used herein, the singular forms “a”, “an”, and “the” include plural references unless the context clearly dictates otherwise. For example, reference to “an agent” includes one or more agents and reference to “a subject” or “the subject” includes one or more subjects.

As used herein, the terms “about” or “approximately” refer to within about 25%, preferably within about 20%, preferably within about 15%, preferably within about 10%, preferably within about 5% of a given value or range. It is understood that such a variation is always included in any given value provided herein, whether or not it is specifically referred to.

As used herein, the term “agent” refers to a substance that, when administered to a patient, causes one or more chemical reactions and/or one or more physical reactions and/or or one or more physiologic reactions and/or one or more immunologic reactions in the patient.

As used herein, the term “ameliorate” refers to improve and/or to make better and/or to make more satisfactory.

As used herein, the term “cell” refers to a single cell as well as a plurality of cells or a population of the same cell type or different cell types. Administering an agent to a cell includes in vivo, in vitro and ex vivo administrations or combinations thereof.

As used herein, the term “complex” refers to an association, either direct or indirect, between one or more particles of an agent and one or more target cells. This association results in a change in the metabolism of the target cell. As used herein, the phrase “change in metabolism” refers to an increase or a decrease in the one or more target cells' production of deoxyribonucleic acid (DNA), ribonucleic acid (RNA), one or more proteins, or any post-translational modifications of one or more proteins.

As used herein, the terms “dysregulation”, “dysregulated” refer to situations or conditions when homeostatic controls systems have been disturbed and/or compromised so that one or more metabolic, physiologic or biochemical systems within a subject operate partially or entirely without said homeostatic control systems.

As used herein, the term “effector molecule” refers to a molecule within a subject that can directly or indirectly regulate the metabolic activity of a target cell by increasing or decreasing the production of DNA, RNA, amino-acid sequences and/or by increasing or decreasing any post-translational modifications of one or more proteins.

As used herein, the term “excipient” refers to any substance, not itself an agent, which may be used as a component within a pharmaceutical composition or a medicament for administration of a therapeutically effective amount of the agent to a subject. Additionally or alternatively an excipient may alone, or in combination with further chemical components, improve the handling and/or storage properties and/or to permit or facilitate formation of a dose unit of the agent. Excipients include, but are not limited to, one or more of: a binder, a disintegrant, a diluent, a buffer, a taste enhancer, a solvent, a thickening agent, a gelling agent, a penetration enhancer, a solubilizing agent, a wetting agent, an antioxidant, a preservative, a surface active agent, a lubricant, an emollient, a substance that is added to mask or counteract a disagreeable odor, fragrances or taste, a substance added to improve appearance or texture of the composition and a substance used to form the pharmaceutical compositions or medicaments. Any such excipients can be used in any dosage forms according to the present disclosure. The foregoing classes of excipients are not meant to be exhaustive but are provided merely as illustrative of what a person of skill in the art would know and would also recognize that additional types and combinations of excipients may be used to achieve delivery of a therapeutically effective amount of the agent to a subject through one or more routes of administration.

As used herein, the terms “inhibit”, “inhibiting”, and “inhibition” refer to a decrease in activity, response, or other biological parameter of a disease, disorder or symptom thereof. This can include but is not limited to the complete ablation of the activity, response, condition, or disease. This may also include, for example, a 10% reduction in the activity, response, condition, or disease as compared to the native or control level. Thus, the reduction can be a 10%, 20%, 30%, 40%, 50%, 60%, 70%, 80%, 90%; 100%, or any amount of reduction in between the specifically recited percentages, as compared to native or control levels.

As used herein, the term “subject” refers to any therapeutic target that receives the agent. The subject can be a vertebrate, for example, a mammal including a human. The term “subject” does not denote a particular age or sex. The term “subject” also refers to one or more cells of an organism; an in vitro culture of one or more tissue types, an in vitro culture of one or more cell types; ex vivo preparations; and a sample of biological materials such as tissue and/or biological fluids.

As used herein, the term “medicament” refers to a medicine and/or pharmaceutical composition that comprises the agent and that can promote recovery from a disease, disorder or symptom thereof and/or that can prevent a disease, disorder or symptom thereof and/or that can inhibit the progression of a disease, disorder, or symptom thereof.

As used herein, the term “patient” refers to a subject that is afflicted with a disease or disorder. The term “patient” includes human and veterinary subjects.

As used herein, the term “pharmaceutical composition” means any composition for administration of the agent to a subject in need of therapy or treatment of a disease, disorder or symptom thereof. Pharmaceutical compositions may include additives such as pharmaceutically acceptable carriers, pharmaceutically accepted salts, excipients and the like. Pharmaceutical compositions may also additionally include one or more further active-ingredients such as antimicrobial agents, anti-inflammatory agents, anaesthetics, analgesics, and the like.

As used herein, the term “pharmaceutically acceptable carrier” refers to an essentially chemically inert and nontoxic component within a pharmaceutical composition or medicament that does not inhibit the effectiveness and/or safety of the agent. Some examples of pharmaceutically acceptable carriers and their formulations are described in Remington (1995, The Science and Practice of Pharmacy (19th ed.) ed. A. R. Gennaro, Mack Publishing Company, Easton, Pa.), the disclosure of which is incorporated herein by reference. Typically, an appropriate amount of a pharmaceutically acceptable carrier is used in the formulation to render the formulation isotonic. Examples of suitable pharmaceutically acceptable carriers include, but are not limited to: saline solutions, glycerol solutions, ethanol, N-(1(2, 3-dioleyloxy)propyl)-N,N,N-trimethylammonium chloride (DOTMA), diolesylphosphotidylethanolamine (DOPE), and liposomes. Such pharmaceutical compositions contain a therapeutically effective amount of the agent, together with a suitable amount of one or more pharmaceutically acceptable carriers and/or excipients so as to provide a form suitable for proper administration to the subject. The formulation should suit the route of administration. For example, oral administration may require enteric coatings to protect the agent from degrading within portions of the subject's gastrointestinal tract. In another example, injectable routes of administration may be administered in a liposomal formulation to facilitate transport throughout a subject's vascular system and to facilitate delivery across cell membranes of targeted intracellular sites.

As used herein, the phrases “prevention of” and “preventing” refer to avoiding an onset or progression of a disease, disorder, or a symptom thereof.

As used herein, the terms “production”, “producing” and “produce” refer to the synthesis and/or replication of DNA, the transcription of one or more sequences of RNA, the translation of one or more amino acid sequences, the post-translational modifications of amino acid sequences, the production or functionality of one or more regulatory molecules that can influence the production or functionality of an effector molecule.

As used herein, the terms “promote”, “promotion”, and “promoting” refer to an increase in an activity, response, condition, disease, or other biological parameter. This can include but is not limited to the initiation of the activity, response, condition, or disease. This may also include, for example, a 10% increase in the activity, response, condition, or disease as compared to the native or control level. Thus, the increase in an activity, response, condition, disease, or other biological parameter can be a 10%, 20%, 30%, 40%, 50%, 60%, 70%, 80%, 90%, 100%, or more, including any amount of increase in between the specifically recited percentages, as compared to native or control levels.

As used herein, the term “prophylactic administration” refers to the administration of any composition to a subject, in the absence of any symptom or indication of a disease or disorder, to prevent the occurrence of and/or the progression of the disease or disorder within the subject.

As used herein, the terms “signal molecule”, “signalling molecule” and “regulatory molecule” can be used interchangeably and they refer to a molecule that can directly or indirectly affect the production and/or functionality of an effector molecule. Signal molecules can act as a direct ligand on a target cell or they may influence the levels or functionality of a downstream ligand.

As used herein, the term “target cell” refers to one or more cells that are deleteriously affected, either directly or indirectly, by a dysregulated immune system.

As used herein, the terms “treat”, “treatment” and “treating” refer to obtaining a desired pharmacologic and/or physiologic effect. The effect may be prophylactic in terms of completely or partially preventing an occurrence of a disease, disorder or symptom thereof and/or may be therapeutic in providing a partial or complete amelioration or inhibition of a disease, disorder, or symptom thereof. Additionally, the term “treatment”, refers to any treatment of a disease, disorder, or symptom thereof in a subject and includes: (a) preventing the disease from occurring in a subject which may be predisposed to the disease but has not yet been diagnosed as having it; (b) inhibiting the disease, i.e., arresting its development; and (c) ameliorating the disease.

As used herein, the term “therapeutically effective amount” refers to the amount of the agent used that is of sufficient quantity to ameliorate, treat and/or inhibit one or more of a disease, disorder or a symptom thereof. The “therapeutically effective amount” will vary depending on the agent used, the route of administration of the agent and the severity of the disease, disorder or symptom thereof. The subject's age, weight and genetic make-up may also influence the amount of the agent that will be a therapeutically effective amount.

As used herein, the terms “unit dosage form” and “unit dose” refer to a physically discrete unit that is suitable as a unitary dose for patients. Each unit contains a predetermined quantity of the agent and optionally, one or more suitable pharmaceutically acceptable carriers, one or more excipients, one or more further active-ingredients, or combinations thereof. The amount of agent within each unit is a therapeutically effective amount.

In one embodiment of the present disclosure, the pharmaceutical compositions disclosed herein comprise an agent as described above in a total amount by weight of the composition of about 0.1% to about 95%. For example, the amount of the agent by weight of the pharmaceutical composition may be about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, about 2.1%>, about 2.2%, about 2.3%, about 2.4%, about 2.5%, about 2.6%, about 2.7%, about 2.8%, about 2.9%, about 3%, about 3.1%, about 3.2%, about 3.3%, about 3.4%, about 3.5%, about 3.6%, about 3.7%, about 3.8%, about 3.9%, about 4%, about 4.1%, about 4.2%, about 4.3%, about 4.4%, about 4.5%, about 4.6%, about 4.7%, about 4.8%, about 4.9%, about 5%, about 5.1%, about 5.2%, about 5.3%, about 5.4%, about 5.5%, about 5.6%, about 5.7%, about 5.8%, about 5.9%, about 6%, about 6.1%, about 6.2%, about 6.3%, about 6.4%, about 6.5%, about 6.6%, about 6.7%, about 6.8%, about 6.9%, about 7%, about 7.1%, about 7.2%, about 7.3%, about 7.4%, about 7.5%, about 7.6%, about 7.7%, about 7.8%, about 7.9%, about 8%, about 8.1%, about 8.2%, about 8.3%, about 8.4%, about 8.5%, about 8.6%, about 8.7%, about 8.8%, about 8.9%, about 9%, about 9.1%, about 9.2%, about 9.3%, about 9.4%, about 9.5%, about 9.6%, about 9.7%, about 9.8%, about 9.9%, about 10%, about 11%, about 12%, about 13%, about 14%, about 15%, about 16%, about 17%, about 18%, about 19%, about 20%, about 25%, about 30%, about 35%, about 40%, about 45%, about 50%, about 55%, about 60%, about 65%, about 70%, about 75%, about 80%, about 85%, about 90% or about 95%.

Where a range of values is provided herein, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also, encompassed within the disclosure, subject to any specifically excluded limit in the stated range. Where the stated range includes one or both of the limits, ranges excluding either or both of those included limits are also included in the disclosure.

The present disclosure relates to one or more agents, therapies, treatments, and methods of use of the agents and/or therapies and/or treatments for upregulating production and/or functionality of one or more immune checkpoint molecules. Some embodiments of the present disclosure relate to methods for making a complex between at least one particle of an agent and at least one target cell of a subject for upregulating the subject's production and/or functionality of one or more immune checkpoint molecules. Embodiments of the present disclosure can be used as a therapy or a treatment for a subject that has a condition whereby the subject's immune system is, or is likely to become, dysregulated.

In some embodiments of the present disclosure, a condition will or is likely to cause the subject's immune system to become dysregulated. The condition may be one of: (i) an autoimmune disease including but not limited to autoimmune atrophic gastritis, autoimmune hemolytic anemia, Addison's disease, asthma, autoimmune uveitis, chronic action hepatitis, connective tissue disease, Crohn's disease, lupus erythematosus, good pasture's syndrome, Graves' disease, Guillain-Barr syndrome, Hashimoto's thyroiditis, idiopathic thrombocytopenia, insulin-dependent diabetes, juvenile idiopathic arthritis myasthenia gravis, migraines, ophthalmia, pemphigus, pernicious anemia, primary myxedema, primary biliary cirrhosis, psoriasis, multiple sclerosis, ulceratis colitis, Sjogren's syndrome, rheumatoid arthritis, polymyositis, scleroderma; idiopathic pulmonary fibrosis (IPF); pulmonary sarcoidosis (ii) a comorbidity with an autoimmune disease including but not limited to chronic fatigue, gastritis, interstitial lung disease, migraines, Raynaud's syndrome, immunodeficiency; (iii) host versus graft disease; (iv) an immune response caused by a surgically implanted foreign body such as an implanted medical device or an implanted diagnostic device; (v) an immune response caused by receiving an isogenic graft or transplant an immune response caused by receiving an allogenic graft or transplant; and (vi) an immune response caused by receiving a xenogenic graft or transplant and combinations thereof.

In some embodiments of the present disclosure, the agent can be administered to the subject by an intravenous route, an intramuscular route, an intraperitoneal route, an intrathecal route, an intravesical route, a topical route, an intranasal route, a transmucosal route, a pulmonary route, and combinations thereof.

In some embodiments of the present disclosure, the agent can be administered to the subject by pipetting a dose of the agent into an in vitro cell culture; perfusing or immersing an ex vivo cell or tissue preparation with a solution that comprises the agent; mixing a biological fluid sample with a solution or substrate that comprises the agent, or combinations thereof.

Some embodiments of the present disclosure relate to an agent that can be administered to a subject with the condition. When a therapeutically effective amount of the agent is administered to the subject, the subject may change production and/or functionality of one or more immune-system molecules. For example, the subject may increase or decrease production and/or functionality of one or more immune-system signaling molecules and/or one or more immune-system effector molecules by changing the production of one or more sequences of DNA, one or more sequences of RNA and/or one or more proteins and/or one or more regulatory molecules that regulate the subject's levels and/or functionality of the signaling immune-system molecules and/or the immune-system effector molecules.

In some embodiments of the present disclosure, the subject may respond to receiving the therapeutic amount of the agent by changing production and/or functionality of one or more intermediary molecules by changing production of one or more DNA sequences, one or more RNA sequences, and/or one or more proteins that regulate the levels and/or functionality of the one or more intermediary molecules. The one or more intermediary molecules regulate the subject's levels and/or functionality of the one or more immune-system signaling molecules and/or the one or more immune-system effector molecules.

In some embodiments of the present disclosure, administering a therapeutic amount of the agent to a subject upregulates the production, functionality or both of one or more checkpoint proteins (CPP). The agent can upregulate production of the one or more CPP by increasing one or more of: synthesis of one or more nucleotides, nucleosides, sequences or genes that are related to increased amounts or functionality of CPP; transcription of RNA that is related to increased amounts or functionality of CPP; or translation of one or more amino acids or amino acid sequences that are related to increased amounts or functionality of CPP. Examples of CPP that the agent can upregulate the production or functionality of include, but are not limited to: cytotoxic T-lymphocyte associated protein 4 (CTLA-4), programmed cell death protein 1 (PD-1), programmed death ligand 1 (PD-L1), programmed death ligand 2 (PD-L2), indoleamine 2, 3-dioxygenase 1 (IDO1), and combinations thereof.

In some embodiments of the present disclosure, administering a therapeutic amount of the agent to a subject upregulates the production, functionality or both of one or more regulatory molecules that regulates the production or functionality of one or more CPP. The one or more regulatory molecules can be a sequence of DNA, RNA or amino acids that causes a decrease or increase in the production or functionality of one or more CPP after administration of the agent. The agent can upregulate production or functionality of the one or more regulatory molecules by increasing one or more of: synthesis of one or more nucleotides, nucleosides, sequences or genes that are related to stimulating or otherwise causing increased amounts or functionality of the one or more regulatory molecules; transcription of RNA that is related to increased amounts or functionality of the one or more regulatory molecules; or translation of one or more amino acids or amino acid sequences that are related to stimulating or otherwise causing increased amounts or functionality of the one or more regulatory molecules. Examples of such regulatory molecules are a sequence of DNA or a sequence of RNA that causes increased amounts or functionality of y interferon.

In some embodiments of the present disclosure, the agent can be: a vector used for gene therapy; one or more selected nucleotides, a sequence of nucleotides, one or more nucleosides, a sequence of nucleosides, a DNA complex, one or more amino acids, a sequence of amino acids, a live microorganism, an attenuated microorganism, a dead microorganism, a recombinant virus, a non-recombinant virus, or combinations thereof.

In some embodiments of the present disclosure, the agent is a vector used for gene therapy. The gene therapy is useful for increasing the production of one or more regulatory molecules and/or one or more CPP. For example, the vector can contain a gene that causes increased expression of y interferon, CTLA-4, PD-1, PD-L1, PD-L2, IDO1 and combinations thereof.

In some embodiments of the present disclosure, the agent is a virus that can be within one or more of the following genus: flavivirus, influenza, enterovirus, rotavirus, rubellavirus, rubivirus, morbillivirus, orthopoxvirus, varicellovirus, dependoparvovirus, alphabaculovirus, betabaculovirus, deltabaculovirus, gammabaculovirus, mastadenovirus, simplexvirus, varicellovirus, cytomegalovirus, or combinations thereof. In some embodiments of the present disclosure the virus is an attenuated virus.

The embodiments of the present disclosure also relate to administering a therapeutically effective amount of the agent. The therapeutically effective amount of the agent will not substantially increase any deleterious conditions within the subject. For example, the therapeutically effective amount will not cause cytokinesis, hypercytokinemia, or any other uncontrolled, or partially controlled, upregulation of the subject's immune system. In some embodiments of the present disclosure, the therapeutically effective amount of the agent that is administered to a patient is between about 10 and about 1×10¹⁶ TCID₅₀/kg (50% tissue culture infective dose per kilogram of the patient's body weight). In some embodiments of the present disclosure the therapeutically effective amount of the agent that is administered to the patient is about 1×10¹³ TCID₅₀/kg. In some embodiments of the present disclosure, the therapeutically effective amount of the agent that is administered to a patient is measured in TPC/kg (total particle count of the agent per kilogram of the patient's body weight). In some embodiments the therapeutically effective amount of the agent is between about 10 and about 1×10¹⁶ TCP/kg.

Some embodiments of the present disclosure relate to a method for making a complex within a subject. The method comprises a step of administering a therapeutically effective amount of the agent to the subject. The complex comprises at least one particle of agent and one or more target cells. When the complex is formed, it affects a change in metabolism of the one or more target cells so that results in the subject upregulating the production and/or functionality of one or more CPP. Examples of a target cell include, but are not limited to: an adrenal gland cell; a B cell; a bile duct cell; a chondrocyte; a cochlear cell; a corneal cell; an endocardium cell; an endometrial cell; an endothelial cell; an epithelial cell; an eosinophil; a fibroblast; a hair follicle cell; a hepatocyte; a lymph node cell; a macrophage; a mucosal cell; a myocyte; a neuron; a glomeruli cell; an optic nerve cell; an osteoblast; an ovarian tissue cell; a pancreatic islet beta cell; a pericardium cell; a platelet; a red blood cell (RBC); a retinal cell; a scleral cell; a Schwann cell; a T cell; a testicular tissue cell; a thyroid gland cell; a uveal cell; or combinations thereof.

Some embodiments of the present disclosure relate to a therapy that can be administered to a subject with the condition. The therapy comprises a step of administering to the subject a therapeutically effective amount of an agent that will upregulate production or activity of one or more regulatory molecules and/or one or more CPP. When the therapy is administered to a patient, the therapy will promote the in vivo production and/or functionality of one or more regulatory molecules and/or one or more CPP. The increased production and/or functionality of the CPP may reduce deleterious effects of the condition upon the patient.

Some embodiments of the present disclosure relate to a method of treating a condition where the method comprises a step of administering to the subject a therapeutically effective amount of an agent that will upregulate production or activity of one or more regulatory molecules and/or one or more CPP.

### Example 1

A patient in her late 20's and of Caucasian ancestry had an extended history of debilitating migraines. The migraines had a typical frequency of three or more episodes per week. The patent also had concurrent asthma symptoms. The patient responded poorly to all existing migraine medications.

The patient was hospitalized for about 30 days after being diagnosed with Guillain-Barr Syndrome (GBS) that had resulted from a viral infection. During the active period when symptoms of GBS were present, the patient's migraines ceased. Subsequent to the symptoms of GBS subsiding, the migraines returned.

After recovery from GBS, the same patient contracted a common rhinovirus infection. During the active period of the virus when the viral symptoms were present, the patient's migraines ceased. Subsequent to the symptoms of the rhinovirus subsiding, the migraines returned.

Subsequently, the same patient contracted the common rhinovirus infection on several occasions. Each time, during the active period when the viral symptoms were present, the patient's migraines ceased. Each time, subsequent to the viral symptoms subsiding, the migraines returned.

### Example 2

A patient in her early 30's and of Caucasian ancestry had an extended history of debilitating migraines with a frequency of about two and half episodes per week with concurrent asthma. Existing migraine medications did not reduce the frequency of migraine episodes. This patient contracted a common upper respiratory viral infection. During the active period of viral symptoms, which lasted about nine days, the patient's migraines ceased. Subsequent to the viral symptoms subsiding, the migraine episodes returned.

### Example 3

A patient in his early 60's and of Caucasian ancestry had a history of migraine aura episodes characterized by sound and light sensitivity and visual distortion. The patient experienced aura episodes at a frequency of about two episodes per week with concurrent asthma. The patient contracted a common gastrointestinal viral infection. During the active period of viral symptoms, which lasted about 14 days, the patient's migraine auras ceased. Subsequent to the viral symptoms subsiding, the migraine aura episodes returned.

1. A method of making an agent/target cell complex, the method comprising a step of administering a sufficient amount of an agent to a target cell whereby the agent/target cell complex is formed, wherein the agent/target cell complex increases the production and/or functionality of one or more checkpoint protein(s) (CPP) by said target cell.

2. The method of claim 1, wherein the step comprises administering a therapeutically effective amount of the agent to a subject, wherein the agent/target cell complex is formed and thereby increasing production and/or functionality of one or more CPP by said target cell.

3. The method of claim 1, wherein the checkpoint protein is cytotoxic T-lymphocyte associated protein 4 (CTLA-4), programmed cell death protein 1 (PD-1), programmed death ligand 1 (PD-L1), programmed death ligand 2 (PD-L2), indoleamine 2, 3-dioxygenase 1 (IDO1) and combinations thereof.

4. The method of claim 1, wherein the agent is at least one of a vector used for gene therapy; one or more selected nucleotides, a sequence of nucleotides, one or more nucleosides, a sequence of nucleosides, a DNA complex, one or more amino acids, a sequence of amino acids, a live microorganism, an attenuated microorganism, a dead microorganism, a recombinant virus, a non-recombinant virus, and combinations thereof.

5. The method of claim 1, wherein the target cell is one or more of an adrenal gland cell; a B cell; a bile duct cell; a chondrocyte; a cochlear cell; a corneal cell; an endocardium cell; an endometrial cell; an endothelial cell; an epithelial cell; an eosinophil; a fibroblast; a hair follicle cell; a hepatocyte; a lymph node cell; a macrophage; a mucosal cell; a myocyte; a neuron; a glomeruli cell; an optic nerve cell; an osteoblast; an ovarian tissue cell; a pancreatic islet beta cell; a pericardium cell; a platelet; a red blood cell (RBC); a retinal cell; a scleral cell; a Schwann cell; a T cell; a testicular tissue cell; a thyroid gland cell; a uveal cell; and combinations thereof.

6. A pharmaceutical agent comprising:
a. an agent that upregulates production or functionality of a checkpoint protein, and/or a regulatory molecule that upregulates the production of or functionality of a checkpoint protein;
b. a pharmaceutically acceptable carrier; and/or
c. an excipient.

7. The pharmaceutical agent according to claim 6, wherein the checkpoint protein is cytotoxic T-lymphocyte associated protein 4 (CTLA-4), programmed cell death protein 1 (PD-1), programmed death ligand 1 (PD-L1), programmed death ligand 2 (PD-L2), indoleamine 2, 3-dioxygenase 1 (IDO1) and combinations thereof.

8. The pharmaceutical agent according to claim 6, wherein the pharmaceutical agent is in a solid form or a liquid form.

9. A method of treating a condition arising from a dysregulated immune system, the method comprising a step of administering to a subject a therapeutically effective amount of an agent that upregulates a production or a functionality of a checkpoint protein.

10. The method according to claim 9, wherein the condition is an autoimmune disease.

11. The method according to claim 9, wherein the condition is a co-morbidity with an autoimmune disease.

12. The method according to claim 9, wherein the condition is an immune-based response to a graft, a transplant or a surgically implanted foreign body.

13. The method according to claim 9, wherein the step of administering occurs by an intravenous route, an intramuscular route, an intraperitoneal route, an intrathecal route, an intravesical route, a topical route, an intranasal route, a transmucosal route, a pulmonary route, and combinations thereof.

14. The method according to claim 9, wherein the checkpoint protein is cytotoxic T-lymphocyte associated protein 4 (CTLA-4), programmed cell death protein 1 (PD-1), programmed death ligand 1 (PD-L1), programmed death ligand 2 (PD-L2), indoleamine 2, 3-dioxygenase 1 (IDO1) and combinations thereof.

15. The method according to claim 9, wherein the agent upregulates a production or a functionality of two or more checkpoint proteins.

16. The method according to claim 9, wherein the agent upregulates a production or a functionality of a regulatory molecule that upregulates the production of or the functionality of a checkpoint protein.

17. The method according to claim 9, wherein the regulatory molecule is a sequence of DNA and/or a sequence of RNA that increases a production of y interferon.

18. The method according to claim 9, wherein the agent is a vector containing a gene for increasing expression of y interferon, CTLA-4, PD-1, PD-L1, PD-L2, IDO1, and combinations thereof.

19. The method according to claim 9, wherein the agent is a virus.

20. The method according to claim 19, wherein the virus is an attenuated virus.

21. The method according to claim 19, wherein the virus is of a genus that is one of a flavivirus, an influenza, an enterovirus, a rotavirus, a rubellavirus, a rubivirus, a morbillivirus, an orthopoxvirus, a varicellovirus, a dependoparvovirus, an alphabaculovirus, a betabaculovirus, a deltabaculovirus, a gammabaculovirus, a mastadenovirus, a simplexvirus, a varicellovirus, a cytomegalovirus, and combinations thereof.

22. The method according to claim 9, wherein the therapeutically effective amount is between about 10 to about 1×10¹⁶ TCID₅₀/kg of the patient's body weight.

23. The method according to claim 9, wherein the therapeutically effective amount is between about 10 to about 1×10¹⁶ total particles of the agent.
